SEQUENCE LISTING (6 sequences)

SEQ ID NO: 1 - DNA, 24 bp, Artificial Sequence
  Description of Artificial Sequence CP-1
cacgaaactaccttcaactccatc

SEQ ID NO: 2 - DNA, 21 bp, Artificial Sequence
  Description of Artificial Sequence CP-2
tgccgacaggatgcagaagga

SEQ ID NO: 3 - DNA, 78 bp, Artificial Sequence
  Description of Artificial Sequence first probe
gcatatagatatctccggcgcggatactttgtgataccgggagttcgcccttataacgtctttttttttttttttttt

SEQ ID NO: 4 - DNA, 18 bp, Artificial Sequence
  Description of Artificial Sequence poly dT primer
tttttttttttttttttt

SEQ ID NO: 5 - DNA, 60 bp, Artificial Sequence
  misc_feature (1): Cy3 attached at the 5′end
cgccggagatatctatatgcccggtatcacaaagtatccggacgttataagggcgaactc

SEQ ID NO: 6 - DNA, 60 bp, Artificial Sequence
  misc_feature (1): Cy3 attached at the 5′end
gcatatagatatctccggcgcggatactttgtgataccgggagttcgcccttataacgtc

(19) United States
(12) Patent Application Publication
(10) Pub. No.: US 2006/0035235 A1
(43) Pub. Date: Feb. 16, 2006

(54) Signal amplification method for detecting expressed gene

(76) Inventors: Mitsugu Usui, Yokohama-shi (JP); Toshihiko Fujikawa, Kanagawa-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/543,683

(22) PCT Filed: Feb. 13, 2004
(86) PCT No.: PCT/JP04/01588

§ 371 Date: Jul. 28, 2005

(30) Foreign Application Priority Data
Feb. 14, 2003 (JP) 2003-037212

(51) Int. Cl. C12Q 1/68; C12P 19/34

(52) U.S. Cl. 435/6; 435/91.2

(57) ABSTRACT

There is provided a signal amplification method for detecting an expressed gene, which allows detection with an inexpensive and simple operation in a short time without expensive enzymes and also allows detection depending on the length or expression amount of the original RNA without using the linear amplification method or the PCR method. In the signal amplification method, the detection sensitivity of the expressed gene on a DNA chip is improved by the use of a reverse transcription reaction and a self-assembly reaction forming a self-assembly substance by means of self-assembling of oligonucleotide probes.

### TECHNICAL FIELD

The present invention relates to a signal amplification method for detecting an expressed gene and more specifically relates to a signal amplification method for detecting an expressed gene which, by the use of a self-assembly reaction of an oligonucleotide, is capable of improving detection sensitivity and detecting a target gene depending on the length and expression amount of a target RNA.

### BACKGROUND ART

Usually, in a detecting method for expressed genes with DNA chips, using a primer having only poly(dT) or a random primer, by a reverse transcription reaction, a labeled cDNA in which a nucleic acid labeled with a fluorescent material such as Cy3 and Cy5 is incorporated turns to a probe. When very small amounts of samples are used, anti-sense RNA is generally synthesized using a linear amplification method (for example, see “Practical Manual of DNA Microarray” supervised by Yoshihide HAYASHIZAKI, issued by YODOSHA CO., LTD., Dec. 1, 2000, pp. 80-90). The linear amplification method includes: synthesizing a first cDNA strand; then synthesizing a second cDNA strand using three enzymes of RNase H, DNA polymerase I and DNA ligase; and finally performing a transcription reaction in vitro with RNA polymerase to amplify anti-sense RNA. However, the linear amplification method has the disadvantage that it needs various types of expensive enzymes and involves a very complicated operation. If the amount of samples is extremely small, the linear amplification method must be performed twice or more. There is also a problem that the anti-sense RNA produced by the linear amplification method tends to be shorter than the original RNA so that the length of the original RNA cannot precisely be reflected.

The present inventors have reported a new isothermal nucleic acid amplification method without using any enzyme (for example, see U.S. Pat. No. 6,261,846, JP 3267576, and EP 1,002,877A). This method uses a pair of oligonucleotides each comprising three regions (Honeycomb Probe, hereinafter referred to as an HCP) in which the three respective regions of a first HCP and a second HCP are designed to be composed of base sequences complementary to each other so that only one region of the first HCP may be hybridized with one region of the second HCP when the two oligonucleotides are reacted. This design makes it possible for a plurality of pairs of the HCPs to hybridize to each other and form an assembly substance by a self-assembly reaction of the HCPs when they are reacted to each other (Probe Alternation Link Self-Assembly Reaction; this method for the formation of an assembly substance by the self-assembly reaction of the HCPs is referred to as a PALSAR method hereinafter).

### DISCLOSURE OF THE INVENTION

It is an object of the present invention to provide a signal amplification method for detecting an expressed gene, which can realize detection with an inexpensive and simple operation in a short time without expensive enzymes and can realize detection depending on the length and expressed amount of the original RNA without the use of the linear amplification method or the PCR method.

In order to solve the above problem, a first aspect of a signal amplification method for detecting an expressed gene according to the present invention is characterized in that the detection sensitivity of the expressed gene on a DNA chip, a DNA microarray, a microwell, or a spherical bead (in the present invention, a DNA chip, a DNA microarray, a microwell, or a spherical bead is generically referred to as “a DNA chip”) is improved by the use of a reverse transcription reaction and a self-assembly reaction forming a self-assembly substance by means of self-assembling of oligonucleotide probes.

A second aspect of a signal amplification method for detecting an expressed gene according to the present invention is characterized in that the detection sensitivity of the expressed gene on a DNA chip is improved by the use of a reverse transcription reaction and a self-assembly reaction forming a self-assembly substance by means of self-assembling of oligonucleotide probes, the signal amplification method comprising the steps of:

performing a reverse transcription reaction of mRNA using a first probe containing poly(dT) at the 3′ end and a region hybridizable with the oligonucleotide probe as a primer to form a second probe having a cDNA region;

separating the mRNA from the second probe;

hybridizing the second probe with a capture probe having a region complementary to a cDNA region of a target mRNA; and

forming a self-assembly substance by a self-assembly reaction using the second probe and the oligonucleotide probe.

While any special limitation is not put on the stage where the step for forming the self-assembly substance by the self-assembly reaction, the forming step is preferably carried out after the step for hybridizing the second probe with the capture probe.

In one aspect, the self-assembly reaction may be performed using a plurality of pairs of oligonucleotide probes, in which the number of base sequence regions complementary each other is n (n≧3), in such a manner that by hybridizing the oligonucleotide probes each other in alternation, the oligonucleotide probes are self-assembled to form a double-stranded self-assembly substance.

In another aspect, the self-assembly reaction may comprise the steps of

providing a first group and a second group,

the first group including a plurality of pairs of dimer-forming probes containing a pair of an oligonucleotide No.1 and an oligonucleotide No.2, each oligonucleotide having three regions of a 3′ side region, a mid-region and a 5′ side region, in which the mid-regions thereof have base sequence complementary to each other to form a dimer probe, and the 3′ side regions and the 5′ side regions thereof have base sequences not complementary to each other, and

the second group including a plurality of pairs of cross-linking probes containing a pair of an oligonucleotide No.3 and an oligonucleotide No.4, each oligonucleotide having two regions of a 3′ side region and a 5′ side region, in which the 3′ side regions and the 5′ side regions thereof have base sequences not complementary to each other, and the pairs of the cross-linking probes having base sequences capable of cross-linking the dimer probes formed from the dimer-forming probes; and

hybridizing the probes,

wherein the oligonucleotide probes are self-asembled to form the self-assembly substance.

The base sequences of the probes may be complementary to each other in the following respective pairs:,

the 3′ side region of the oligonucleotide No.1 in the first group and

the 3′ side region of the oligonucleotide No.3 in the second group;

the 5′ side region of the oligonucleotide No.2 in the first group and

the 5′ side region of the oligonucleotide No.4 in the second group;

the 3′ side region of the oligonucleotide No.4 in the second group and

the 3′ side region of the oligonucleotide No.2 in the first group; and

the 5′ side region of the oligonucleotide No.3 in the second group and

the 5′ side region of the oligonucleotide No.1 in the first group.

The base sequences of the probes may be complementary to each other in the following respective pairs:,

the 3′ side region of the oligonucleotide No.1 in the first group and

the 3′ side region of the oligonucleotide No.3 in the second group;

the 5′ side region of the oligonucleotide No.2 in the first group and

the 5′ side region of the oligonucleotide No.3 in the second group;

the 3′ side region of the oligonucleotide No.2 in the first group and

the 3′ side region of the oligonucleotide No.4 in the second group; and

the 5′ side region of the oligonucleotide No.1 in the first group and

the 5′ side region of the oligonucleotide No.4 in the second group.

A third aspect of a signal amplification method for detecting an expressed gene according to the present invention is characterized in that the detection sensitivity of the expressed gene on a DNA chip is improved by the use of a reverse transcription reaction and a self-assembly reaction, wherein the self-assembly reaction is performed using a plurality of pairs of a first HCP and a second HCP of oligonucleotide probes, in which the number of base sequence regions complementary each other is n (n≧3), in such a manner that by hybridizing the oligonucleotide probes each other in alternation, the oligonucleotide probes are self-assembled to form a double-stranded self-assembly substance, the signal amplification method comprising the steps of:

binding a first probe containing poly(dT) at the 3′ end and at least a part of the base sequence regions of the first HCP to mRNA;

performing a reverse transcription reaction by a reverse transcriptase to form a second probe containing a cDNA region and at least a part of the base sequence regions of the first HCP;

removing the mRNA; thereafter

hybridizing the second probe with a capture probe having a region complementary to a cDNA region of a target mRNA; and

adding both the first HCP and the second HCP or adding the second HCP to form a self-assembly substance by the self-assembly reaction of the oligonucleotide probes so that signal amplification can be achieved.

In the signal amplification method of the present invention, there may be employed mRNA containing poly(A) at the end thereof as a target expressed gene.

It is preferable that the DNA chip has a support to which the capture probe for capturing the target gene is bound and the support is a microplate type, a slide glass type, a particle type, or an electroconductive substrate type. The support of the microplate type or the particle type may be made of plastics such as polystyrene. Materials such as glass or plastics may be employed for the support of the slide glass type. A gold electrode, an ITO (indium oxide) electrode or the like may be used for the support of the electroconductive substrate type.

A labeled probe may be hybridized with the self-assembly substance so that the presence of the self-assembly substance can be detected.

The labeled probe is preferably a probe labeled with an enzyme of color generation type, an enzyme of luminescence generation type or a radioisotope.

The presence of the self-assembly substance may be detected by:

adding a fluorescent substance capable of binding to a nucleic acid to the self-assembly substance; and

measuring a photochemical change of the fluorescent substance.

The presence of the self-assembly substance may be detected by:

labeling in advance at least one of the oligonucleotide probes forming the self-assembly substance with a fluorescent substance; and

measuring a photochemical change of the fluorescent substance.

The presence of the self-assembly substance may be detected by:

labeling in advance at least one of the oligonucleotide probes forming the self-assembly substance with a radioisotope; and

detecting the radioisotope.

The presence of the self-assembly substance may be detected by:

labeling in advance at least one of the oligonucleotide probes forming the self-assembly substance with an enzyme of color generation type or an enzyme of luminescence generation type; and

measuring a photochemical change due to the enzyme.

The oligonucleotide probes may be comprised of at least one base selected from the group consisting of DNA, RNA, PNA, and LNA.

### BRIEF DESCRIPTION OF THE DRAWINGS

FIG. 1 is a flow chart showing an example of order of steps of a signal amplification method according to the present invention;

FIG. 2 is a schematic diagram showing in principle the step 200 in a first example of order of steps of the signal amplification method according to the present invention;

FIG. 3 is a schematic diagram showing in principle the step 202 in the first example of order of steps of the signal amplification method of the present invention;

FIG. 4 is a schematic diagram showing in principle the step 204 in the first example of order of steps of the signal amplification method of the present invention;

FIG. 5 is a schematic diagram showing in principle the step 206 in the first example of order of steps of the signal amplification method of the present invention;

FIG. 6 is a schematic diagram showing in principle the step 210 in the first example of order of steps of the signal amplification method of the present invention;

FIG. 7 is a schematic diagram showing in principle the step 212 in the first example of order of steps of the signal amplification method of the present invention;

FIG. 8 is a schematic diagram showing in principle the step 214 in the first example of order of steps of the signal amplification method of the present invention;

FIG. 9 is a schematic diagram showing in principle the step 300 in a second example of order of steps of the signal amplification method of the present invention;

FIG. 10 is a schematic diagram showing in principle the step 302 in the second example of order of steps of the signal amplification method of the present invention;

FIG. 11 is a schematic diagram showing in principle the step 304 in the second example of order of steps of the signal amplification method of the present invention;

FIG. 12 is a schematic diagram showing in principle the step 306 in the second example of order of steps of the signal amplification method of the present invention; and

FIG. 13 is a graph showing the results of Example 1 and Comparative Example 1.

### BEST MODE FOR CARRYING OUT THE INVENTION

The examples of the present invention are described below with reference to the attached drawings. It should be understood that the examples described herein are merely exemplary and that many variations and modifications may be made without departing from the spirit and scope of the present invention.

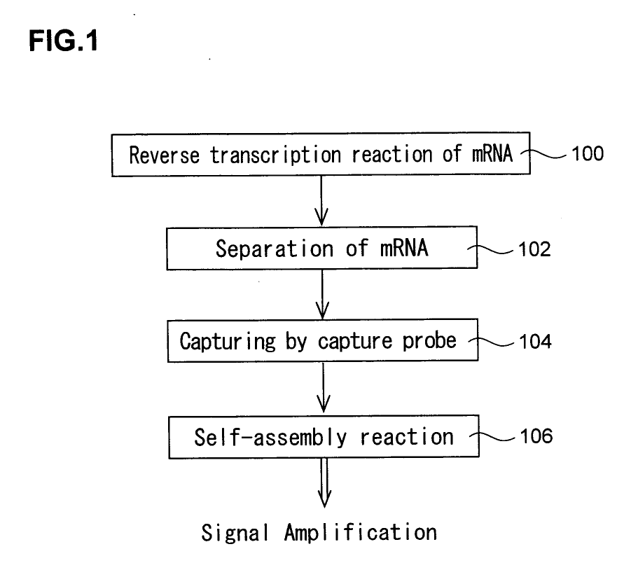

FIG. 1 is a flow chart showing an example of order of steps of the signal amplification method for detecting an expressed gene according to the present invention.

As shown in FIG. 1, there is provided a first probe which contains poly(dT) at the 3′ end and a region hybridizable with at least one oligonucleotide probe for use in a self-assembly reaction. The first probe is used as a primer and bound to mRNA containing poly(A). A reverse transcription reaction of the mRNA is performed by a reverse transcriptase (step 100) so that there is formed a second probe which is comprised of the first probe and a cDNA region of the mRNA. In a preferred mode, a base sequence in the 5′ side of the first probe includes at least a part of the base sequence of the oligonucleotide probe for use in the self-assembly reaction.

The mRNA is then separated from the second probe (step 102). Any limitations are not specifically put on the method for separating the mRNA, but methods using, for example, thermal denaturation, alkali denaturation, RNA digestion with RNase H, or the like may be employed.

After the separation, the second probe is hybridized with a capture probe having a region complementary to a CDNA region of a target mRNA to allow the capture probe to capture the second probe (step 104). Preferably, the capture probe has previously been bound to a support.

The oligonucleotide probes are added so that a self-assembly substance hybridized with the second probe can be formed by a self-assembly reaction (step 106) with the result that the signal can be amplified.

If the target mRNA does not exist in the sample, the second probe cannot bind to the capture probe so that signal amplification cannot be carried out. Thus, the existence of the target mRNA can be determined by the signal amplification method of the present invention. Since the signal amplification method of the present invention does not use the linear amplification method, detection can be performed depending on the length of the original RNA, and since the signal amplification method of the present invention does not use the PCR method, signal amplification can be performed depending on the amount of expression.

As the self-assembly reaction described above, there may be employed a self-assembly reaction using a pair of HCPs where each of the HCPs comprises three regions complementary to each other and the HCPs can self-assemble by themselves to form a self-assembly substance (for example, see JP 3267576 and JP 3310662). Alternatively, a self-assembly reaction may be performed using a pair of dimer-forming probes capable of forming a dimer by themselves and a pair of crosslinking probes capable of crosslinking the dimer formed from the dimer-forming probes (for example, see JP-A 2002-355081).

Although step 104 is followed by step 106 in the example shown in FIG. 1, step 106 may be performed before or simultaneously with step 104.

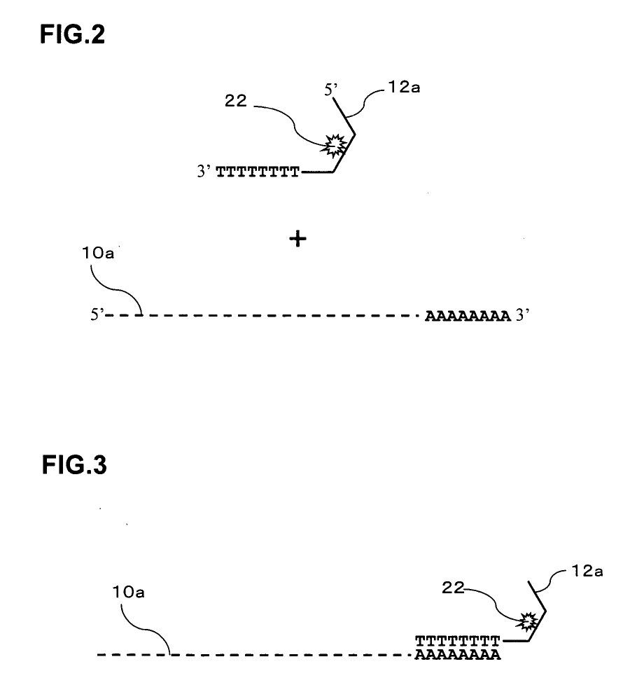

FIGS. 2 to 8 are schematic diagrams showing in principle a first example of order of steps of the signal amplification method according to the present invention. The first example illustrates the signal amplification method utilizing the PALSAR method using a pair of HCPs previously labeled with a fluorescent material 22 as the self-assembly reaction, wherein an HCP containing poly(dT) at the 3′ end is used as a first probe 12a.

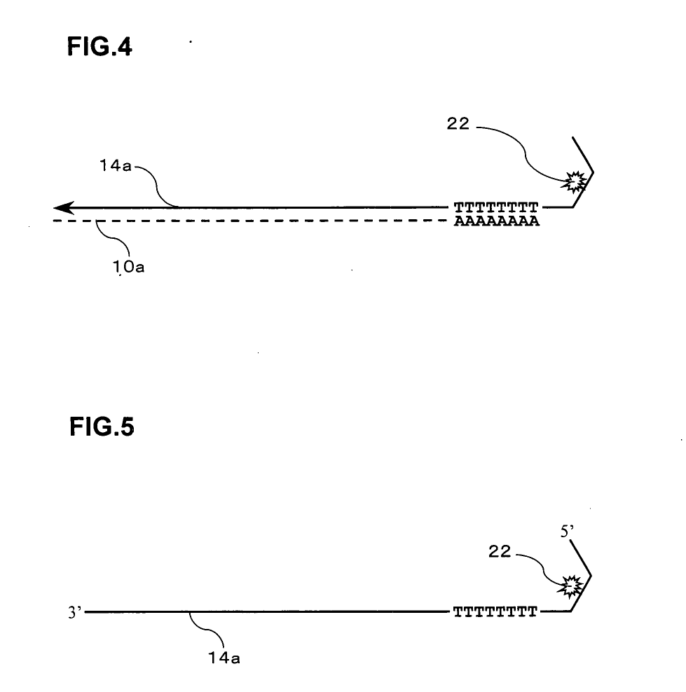

As shown in FIG. 2, in order to detect mRNA 10a of a target gene, an oligonucleotide probe (HCP-1) containing poly(dT) at the 3′ end and three regions of the HCP in the 5′ side thereof is provided as the first probe 12a (step 200). As shown in FIG. 3, HCP-1 (12a) containing poly(dT) at the 3′ end is bound to the poly(A) tail part of the mRNA 10a (step 202). As shown in FIG. 4, a reverse transcription reaction is then performed using a reverse transcriptase to form a second probe 14a having a sequence complementary to the mRNA (step 204). Thereafter, as shown in FIG. 5, the mRNA 10a is dissociated to form a single-stranded oligonucleotide comprising a cDNA region and the HCP region (step 206).

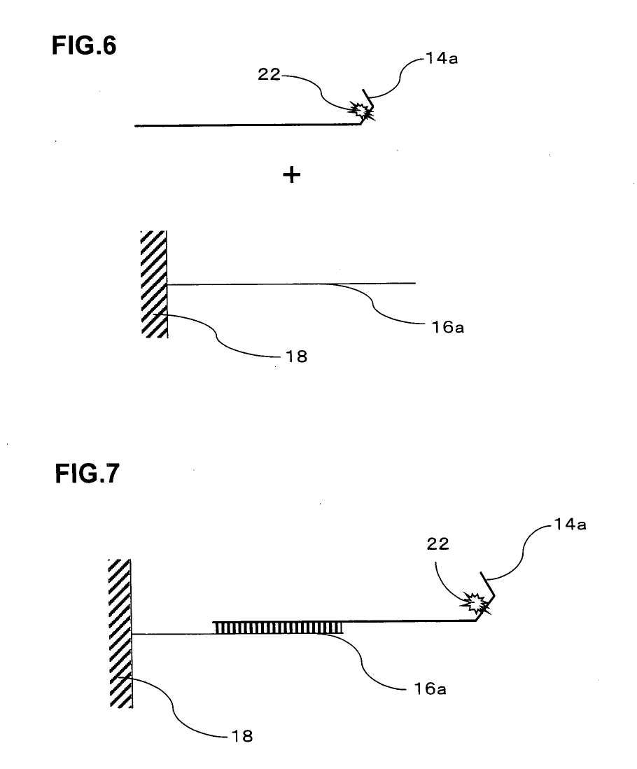

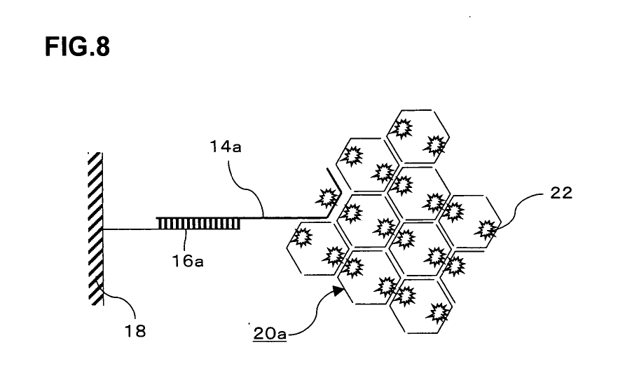

As shown in FIG. 6, a capture probe 16a having a region complementary to the cDNA of the target gene is previously bound to a support 18 (step 210). As shown in FIG. 7, the second probe 14a serving as an HCP having the formed cDNA region is hybridized with the capture probe 16a (step 212). As shown in FIG. 8, another HCP (HCP-2) of the pair of HCPs is added to form a self-assembly substance 20a by a self-assembly reaction (step 214) so that signal amplification can be achieved. Incidentally, if in the step 206, a washing operation is carried out when the target mRNA is removed, the pair of HCPs must be added in the step 214.

In the above first example, there is used a pair of HCPs, wherein a part of the 3′ side region in the three regions of HCP-1 is poly(dT). Alternatively, there may be used another pair of HCPs, wherein the 3′ side region of HCP-1 is entirely poly(dT) and the 3′ side region in the three regions of HCP-2 is entirely poly(A).

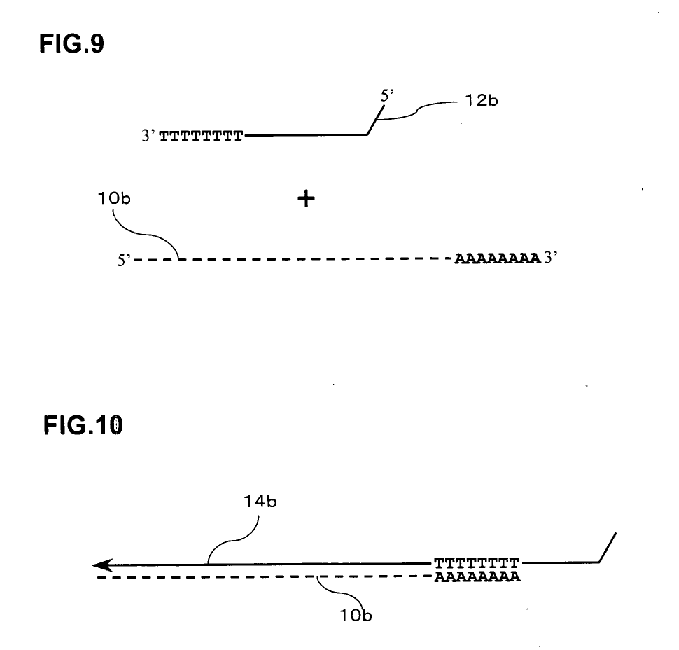

FIGS. 9 to 12 are schematic diagrams showing in principle a first example of order of steps of the signal amplification method according to the present invention. The second example illustrates the signal amplification method utilizing the PALSAR method using a pair of HCPs unlabeled with a fluorescent material as the self-assembly reaction, wherein an oligonucleotide probe containing poly(dT) at the 3′ end and one of complementary regions of the HCP is used as a first probe 12b.

As shown in FIG. 9, in order to detect mRNA 10b of a target gene, an oligonucleotide probe containing poly(dT) at the 3′ end and one of the complementary regions of the HCP in the 5′ side thereof is provided as the first probe 12b (step 300). As shown in FIG. 10, the oligonucleotide probe 12b is bound to the poly(A) tail part of the mRNA 10b, and a reverse transcription reaction is performed using a reverse transcriptase to form a second probe 14b having a sequence complementary to the mRNA (step 302). The mRNA 10b is separated to form a single-stranded oligonucleotide comprising a cDNA region and the HCP region.

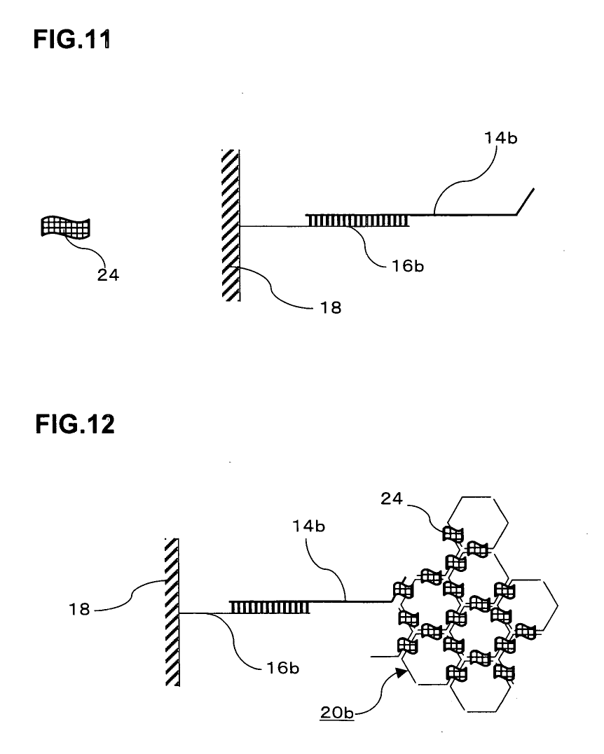

As shown in FIG. 11, the second probe 14b is hybridized with a capture probe 16b bounded to a support 18 (step 304). As shown in FIG. 12, a pair of HCPs is added to form a self-assembly substance 20b by a self-assembly reaction (step 306). An intercalator 24 or the like is inserted into the formed self-assembly substance 20b (step 308) so that signal amplification can be achieved. Steps 306 and 308 may be performed simultaneously.

For detection of the target gene, a labeling material for detection may previously be added to the pair of the oligonucleotide probes. Examples of such a labeling material include radioisotopes such as I¹²⁵ and P³², luminescent materials such as digoxigenin and acridinium esters, fluorescent materials such as Cy3 and Cy5, and fluorescent donor dyes and fluorescent acceptor dyes for using fluorescent resonance energy transfer (FRET) such as biotin for using a fluorescent material such as 4-methylunbelliferyl phosphate.

Alternatively, by adding a dye having the property of binding to nucleic acids, the target gene can be detected. A fluorescent material having the property of binding to nucleic acids, such as an intercalator, is preferably used to detect the target gene. Any fluorescent material having the property of binding to nucleic acids may be used without limitation. Examples of such a fluorescent material include SYBR Green I stain, SYBR Green II stain, SYBR Green Gold stain, Vistra Green stain, Gelstar stain, Radlant Red stain, PicoGreen, RiboGreen, OllGreen, Hoechst 33258 (Bis-Benzimide), Propidium Iodide, YO-PRO-1 Iodide, YO-PRO-3 Iodide (the above materials are all manufactured by Molecular Probes Inc.), ethidium bromide, Distamycin A, TOTO, Psoralen, acridinium orange (Acridine Orange), AOAO (homodimer), and the like.

While a nucleic acid constituting the pair of the oligonucleotide probes is usually DNA or RNA, a nucleic acid analogue may constitute them. Examples of such a nucleic acid analogue include peptide nucleic acids (PNAs, for example, see the brochure of International Patent Publication No. WO 92/20702) and locked nucleic acids (LNAs, for example, see Koshkin AA et al., Tetrahedron 1998, 54, 3607-3630, Koshkin AA et al., J. Am. Chem. Soc., 1998, 120, 13252-13253 and Wahlestedt C et al., PNAS, 2000, 97, 5633-5638). The pair of the oligonucleotide probes is generally composed of nucleic acids of the same kind, but may be composed of a pair of a DNA probe and an RNA probe. That is, the type of the nucleic acid of the probe may be selected from DNA, RNA or nucleic acid analogues (such as PNA and LNA). Also, it is not necessary that a single probe is composed of a single type of nucleic acid, for example, DNA only, and, if necessary, for example, an oligonucleotide probe composed of DNA and RNA (a chimera probe) may be used in an aspect of the present invention.

In terms of the number of bases, the length of each complementary base sequence region of the oligonucleotide probe may be at least 5 bases, preferably from 10 to 100 bases, more preferably from 15 to 30 bases.

These probes may be synthesized by any known methods. For example, DNA probes may be synthesized by a phosphoamidite method using DNA Synthesizer Model 394 (Applied Biosystems Inc.). Any other synthesis methods may also be used such as a phosphotriester method, an H-phosphonate method, and a thiophosphonate method.

According to the present invention, a self-assembly substance is formed with a pair of HCPs having complementary regions against a target gene captured on a DNA chip. While the number of pieces of the oligonucleotide probe for use is not limited, it may be in the range of 10² to 10¹⁵. The reaction buffer solution may have any composition and any concentration, and any buffer solution commonly used for nucleic acid amplification may be preferably used. The pH may be in any conventional range, preferably in the range of 7.0 to 9.0. The reaction temperature may be from 40 to 80° C., preferably from 55 to 65° C.

In the present invention, any sample potentially containing a target nucleic acid may be used as a sample for the measurement of a target expressed gene (mRNA). The target gene may be any properly prepared or isolated from samples and it is not specifically limited. Examples of such samples include organism-derived samples such as blood, blood serum, urine, feces, cerebrospinal fluid, tissue fluid, and cell cultures, and any samples potentially containing or potentially infected with any eukaryote having mRNA containing a poly(A) strand at its end, such as fungi. There may be also used any nucleic acid obtained by amplifying a target gene in samples with any known method.

### EXAMPLES

Although the present invention is more specifically described by means of the examples below, it will be understood that the examples presented are by way of illustration only and should not be construed as any limitation on the present invention.

The materials below were used in the examples.

(a) Target Gene: Total RNA Extracted from Cultured Cells

(b) Capture Probe:

1) CP-1: 5′-CACGAAACTACCTTCAACTCCATC-3′
2) CP-2: 5′-TGCCGACAGGATGCAGAAGGA-3′

(c) Primers:

1) First probe (poly(dT)-HCP, 78 mer): 5′-GCATATAGATATCTCC GGCGCGGATACTTTGTGATACCGGGAGTTCGCCCTTATAACGTCTTTTT TTTTTTTTTTTTT-3′

2) Poly(dT) primer (18 mer): 5′-TTTTTTTTTTTTTTTTTT-3′

(d) HCPs:

1) HCP-1 (Cy3-labeled 5′ end, 60 mer): 5′-Cy3-CGCCGGAGATAT CTATATGCCCGGTATCACAAAGTATCCGGACGTTATAAGGGCGAACTC-3′

2) HCP-2 (Cy3-labeled 5′end, 60 mer): 5′-Cy3-GCATATAGATATC TCCGGCGCGGATACTTTGTGATACCGGGAGTTCGCCCTTATAACGTC-3′

(e) Polystyrene Particle Beads: a Single Kind of Particle Beads Having the Above Two Kinds of Capture Probes Fixed Thereon.

### Example 1

Using the total RNA extracted from cultured cells, an attempt was made to detect beta-actin of a housekeeping gene according to the PALSAR method.

(1) Reverse Transcription Reaction of RNA and Purification of Reverse Transcription Product

A reverse transcription reaction was carried out at 37° C. for 2 hours using the target gene, the first probe, a reverse transcriptase (SuperScript II manufactured by Invitrogen Corporation), and a reaction solution (Reaction Buffer, DTT, dNTP, RNase inhibitor). Thereafter, alkali treatment was performed at 65° C. for 30 minutes, and neutralization was performed using hydrochloric acid. The reverse transcription product of cDNA was purified using QIAquick PCR Purification Kit (manufactured by QIAGEN K. K.).

(2) Hybridization

The obtained cDNA, the particle beads, 6×SSC, 0.2% SDS, and 5× Denhardt's solution, were then mixed to prepare a composition of 50 μl in total amount. The composition was subjected to hybridization at 42° C. for 2 hours. After the hybridization was completed, filtration was performed with a 0.22 μm filter so that the unreacted probe was removed. Then the particle beads was cleaned once with 2×SSC+0.1% SDS and once with 0.2×SSC, and was filtered with the above filter.

Thereafter, a composition of 100 μl in total amount consisting of the obtained particle beads, the HCPs-1 and 2 (each 1.5 pmol/μl), 1% Blocking Reagent (manufactured by Roche Inc.), 0.1% N-lauroylsarcosine, 0.02% SDS, and 5×SSC was subjected to hybridization at 65° C. for 30 minutes.

(3) Detection

After washing, the particle beads were resuspended in sheath fluid for a flow cytometer, and the fluorescence of the Cy3 labeled to the HCPs was measured with the flow cytometer.

### Comparative Example 1

The target gene was detected using a conventional Cy3-dNTP uptake system with a poly(dT) primer.

(1) Reverse Transcription Reaction and Separation Reaction of RNA

The reverse transcription reaction and the purification of the reverse transcription product were performed under the same process as in Example 1 with the exception of using the poly(dT) primer as primer, and introducing Cy3-labeled dUTP (manufactured by Amersham Inc.) in addition to dNTP.

(2) Hybridization

The obtained CDNA, the particle beads, 6×SSC, 0.2% SDS, and 5× Denhardt's solution were then mixed to prepare a composition of 50 μl in total amount. The composition was subjected to hybridization at 42° C. for 2 hours. After the hybridization was completed, filtration was performed with a 0.22 μm filter so that the unreacted probe was removed. Then the composition was cleaned once with 2×SSC+0.1% SDS, and was filtered.

(3) Detection

After washing, the particle beads were resuspended in a sheath fluid for a flow cytometer, and the fluorescence of the Cy3 label of the cDNA bound to the capture probe on the particle beads was measured with the flow cytometer.

[Results]

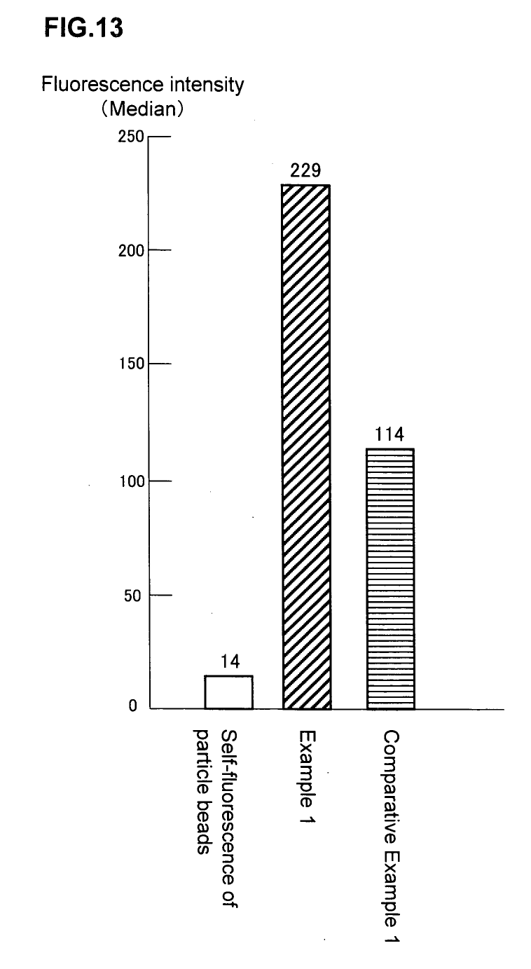

The results of Example 1 and Comparative Example 1 are shown in FIG. 13. The fluorescence intensity was measured for 203 to 504 particle beads in respect of each one and indicated by the median thereof. As shown in FIG. 13, the detection sensitivity of Example 1 is significantly higher than that of Comparative Example 1.

Capability of Exploitation in Industry

As described above, according to the present invention, the detection only through a reverse transcription reaction can be accomplished by an inexpensive and simple operation in a short time without expensive enzymes. A further significant advantage is that since there is no need to use the linear amplification method or the PCR method, the detection can also be performed depending on the length or expression amount of the original RNA.

1. A signal amplification method for detecting an expressed gene, wherein the detection sensitivity of the expressed gene on a DNA chip is improved by the use of a reverse transcription reaction and a self-assembly reaction forming a self-assembly substance by means of self-assembling of oligonucleotide probes.

2. A signal amplification method for detecting an expressed gene, wherein the detection sensitivity of the expressed gene on a DNA chip is improved by the use of a reverse transcription reaction and a self-assembly reaction forming a self-assembly substance by means of self-assembling of oligonucleotide probes, the signal amplification method comprising the steps of:
performing a reverse transcription reaction of mRNA using a first probe containing poly(dT) at the 3′ end and a region hybridizable with the oligonucleotide probe as a primer to form a second probe having a cDNA region;
separating the mRNA from the second probe;
hybridizing the second probe with a capture probe having a region complementary to a cDNA region of a target mRNA; and
forming a self-assembly substance by a self-assembly reaction using the second probe and the oligonucleotide probe.

3. The signal amplification method according to claim 1 or 2, wherein the self-assembly reaction is performed using a plurality of pairs of oligonucleotide probes, in which the number of base sequence regions complementary each other is n (n≧3), in such a manner that by hybridizing the oligonucleotide probes each other in alternation, the oligonucleotide probes are self-assembled to form a double-stranded self-assembly substance.

4. A signal amplification method for detecting an expressed gene, wherein the detection sensitivity of the expressed gene on a DNA chip is improved by the use of a reverse transcription reaction and a self-assembly reaction, wherein the self-assembly reaction is performed using a plurality of pairs of a first HCP and a second HCP of oligonucleotide probes, in which the number of base sequence regions complementary each other is n (n≧3), in such a manner that by hybridizing the oligonucleotide probes each other in alternation, the oligonucleotide probes are self-assembled to form a double-stranded self-assembly substance, the signal amplification method comprising the steps of:
binding a first probe containing poly(dT) at the 3′ end and at least a part of the base sequence regions of the first HCP to mRNA;
performing a reverse transcription reaction by a reverse transcriptase to form a second probe containing a cDNA region and at least a part of the base sequence regions of the first HCP;
removing the mRNA; thereafter
hybridizing the second probe with a capture probe having a region complementary to a cDNA region of a target mRNA; and
adding both the first HCP and the second HCP or adding the second HCP to form a self-assembly substance by the self-assembly reaction of the oligonucleotide probes so that signal amplification can be achieved.

5. The signal amplification method according to claim 1 or 2, wherein
the self-assembly reaction comprises the steps of:
providing a first group and a second group,
the first group including a plurality of pairs of dimer-forming probes containing a pair of an oligonucleotide No.1 and an oligonucleotide No.2, each oligonucleotide having three regions of a 3′ side region, a mid-region and a 5′ side region, in which the mid-regions thereof have base sequence complementary to each other to form a dimer probe, and the 3′ side regions and the 5′ side regions thereof have base sequences not complementary to each other, and
the second group including a plurality of pairs of cross-linking probes containing a pair of an oligonucleotide No.3 and an oligonucleotide No.4, each oligonucleotide having two regions of a 3′ side region and a 5′ side region, in which the 3′ side regions and the 5′ side regions thereof have base sequences not complementary to each other, and the pairs of the cross-linking probes having base sequences capable of cross-linking the dimer probes formed from the dimer-forming probes; and
hybridizing the probes,
wherein the oligonucleotide probes are self-assembled to form the self-assembly substance.

6. The signal amplification method according to claim 5, wherein the base sequences of the probes are complementary to each other in the following respective pairs:,
the 3′ side region of the oligonucleotide No.1 in the first group and
the 3′ side region of the oligonucleotide No.3 in the second group;
the 5′ side region of the oligonucleotide No.2 in the first group and
the 5′ side region of the oligonucleotide No.4 in the second group;
the 3′ side region of the oligonucleotide No.4 in the second group and
the 3′ side region of the oligonucleotide No.2 in the first group; and
the 5′ side region of the oligonucleotide No.3 in the second group and
the 5′ side region of the oligonucleotide No.1 in the first group.

7. The signal amplification method according to claim 5, wherein the base sequences of the probes are complementary to each other in the following respective pairs:,
the 3′ side region of the oligonucleotide No.1 in the first group and
the 3′ side region of the oligonucleotide No.3 in the second group;
the 5′ side region of the oligonucleotide No.2 in the first group and
the 5′ side region of the oligonucleotide No.3 in the second group;
the 3′ side region of the oligonucleotide No.2 in the first group and
the 3′ side region of the oligonucleotide No.4 in the second group; and
the 5′ side region of the oligonucleotide No.1 in the first group and
the 5′ side region of the oligonucleotide No.4 in the second group.

8. The signal amplification method according to any one of claims 2 to 7, wherein the capture probe is bound to a support.

9. The signal amplification method according to claim 8, wherein the support is a microplate type, a slide glass type, a particle type, or an electroconductive substrate type.

10. The signal amplification method according to any one of claims 1 to 9, further comprising hybridizing a labeled probe with the self-assembly substance to detect the presence of the self-assembly substance.

11. The signal amplification method according to claim 10, wherein the labeled probe is a probe labeled with an enzyme of color generation type, an enzyme of luminescence generation type or a radioisotope.

12. The signal amplification method according to any one of claims 1 to 9, wherein the presence of the self-assembly substance is detected by:
adding a fluorescent substance capable of binding to a nucleic acid to the self-assembly substance; and
measuring a photochemical change of the fluorescent substance.

13. The signal amplification method according to any one of claims 1 to 9, wherein the presence of the self-assembly substance is detected by:
labeling in advance at least one of the oligonucleotide probes forming the self-assembly substance with a fluorescent substance; and
measuring a photochemical change of the fluorescent substance.

14. The signal amplification method according to any one of claims 1 to 9, wherein the presence of the self-assembly substance is detected by:
labeling in advance at least one of the oligonucleotide probes forming the self-assembly substance with a radioisotope; and
detecting the radioisotope.

15. The signal amplification method according to any one of claims 1 to 9, wherein the presence of the self-assembly substance is detected by:
labeling in advance at least one of the oligonucleotide probes forming the self-assembly substance with an enzyme of color generation type or an enzyme of luminescence generation type; and
measuring a photochemical change due to the enzyme.

16. The signal amplification method according to any one of claims 1 to 15, wherein the oligonucleotide probes are comprised of at least one base selected from the group consisting of DNA, RNA, PNA, and LNA.
